(19) United States
(12) Patent Application Publication
(10) Pub. No.: US 2021/0113129 A1
(43) Pub. Date: Apr. 22, 2021

(54) A SYSTEM FOR DETERMINING EMOTIONAL OR PSYCHOLOGICAL STATES

(71) Applicant: Sin-Ger Huang, Taipei (CN)

(72) Inventor: Sin-Ger Huang, Taipei (CN)

(21) Appl. No.: 16/464,294

(22) PCT Filed: Nov. 30, 2017
(86) PCT No.: PCT/CN2017/114045

§ 371 Date: May 28, 2019

Related U.S. Application Data
(60) Provisional application No. 62/428,543, filed on Dec. 1, 2016.
(60) Provisional application No. 62/428,544, filed on Dec. 1, 2016.

(51) Int. Cl. A61B 5/16; A61B 5/00; G06F 3/01

(52) U.S. Cl. CPC A61B 5/165; A61B 5/486; G06F 3/011; A61B 5/6803; A61B 5/7221; A61B 5/163

(57) ABSTRACT

A system (100) for determining emotional or psychological state includes a human-multimedia interaction system (102) and a detector (104). The human-multimedia interaction system (102) includes a head mounted apparatus (1021) with a display device (1023), a processing device (1025), and a storage device (2026). The detector (104) detects at least one characteristic of a wearer. The processing device (1025) receives the characteristic of the wearer and compares the characteristic of the wearer with existing data in the storage device (2026) or from the cloud. When the characteristic of the wearer is determined or verified by the processing device (1025), the processing device (1025) transmits at least one of the video content signals, according to the characteristic of the wearer to the display device (1023) and the display device (1023) displays the video content signal.

### FIELD OF THE INVENTION

The present invention relates to a system for determining emotional or psychological states.

### BACKGROUND

Augmented or virtual reality systems can simulate a user's physical presence in visual spaces. Simulations may include a 360° view of the surrounding visual space such that the user may turn his head to watch content presented within the visual space. (Note that the term “he/his” is used generically throughout the application to indicate both male and female.) If augmented or virtual reality contents can be developed or delivered with a system capable of determining emotional or psychological states of the user, the contents will be more affecting and effective.

### SUMMARY

The present invention is directed to a system for determining emotional or psychological states. In a first embodiment, the system includes a human-multimedia interaction system, and a detector. The human-multimedia interaction system includes a head mounted apparatus with a display device, a processing device, and a storage device. The detector detects at least one characteristic of the wearer. The processing device receives the characteristic reading of the wearer and compares the characteristic of the wearer with existing data in the storage device or from the cloud. The storage device may contain cloud-updated or locally collected data.

When the characteristic of the wearer is determined or verified by the processing device, the processing device transmits at least one of the video content signals, according to the characteristic of the wearer to the display device and the display device displays the video content signal.

In a second embodiment, the processing device and the head mounted apparatus include a wireless communication unit, the detector detects at least one characteristic of the wearer, the head mounted apparatus transmits the characteristic of the wearer to the processing device by wireless communication, the processing device compares the characteristic of the wearer with the existing data in the storage device or from cloud and transmits at least one of the video content signals according to the characteristic of the wearer to the head mounted apparatus by wireless communication.

In a third embodiment, the detector is worn on, attached to, or mounted on the wearer, each one of the detector and the head mounted apparatus includes a wireless communication unit, the detector detects at least one the characteristic of the wearer and transmits the characteristic of the wearer to the head mounted apparatus or the process device by wireless communication.

In a fourth embodiment, the detector detects at least one characteristic of the wearer, the processing device compares the characteristic of the wearer detected by the detector with existing date in the storage device or from the cloud and determines at least one emotional or psychological state of the wearer corresponding to the characteristic of the wearer. The processing device transmits at least one the video content signal according to the emotional or psychological state of the wearer to the display device.

In a fifth embodiment, the system is used to communicate with at least one user who wears the head mounted apparatus in an augmented or virtual environment or an internet environment. The processing device determines the wearer's identity or emotional or psychological state and searches for at least one video content signal according to the characteristic of the wearer. The video content signal includes a personal setup signal set by the wearer according to a face parameter and a body parameter of the wearer, the processing device can set a virtual body video signal according to the personal setup signal of the video content signal and transmits the virtual body video signal of the wearer to the head mounted apparatus of the user for communicating with each other in the virtual environment or the internet environment.

In at least one embodiment, the detector can detect the characteristic of the wearer at predefined intervals to observe change of the emotional or psychological state of the wearer, the processing device transmits new video content signal according to the change of the emotional or psychological state of the wearer, and the display device replaces the video content signal with new video content signal.

In at least one embodiment, the detector of the head mounted apparatus detects a change of the face parameter of the wearer like facial expressions, and the processing device receives the change of the face parameter of the wearer for changing a facial expression of the virtual body video signal of the wearer for instance.

### BRIEF DESCRIPTION OF THE DRAWINGS

The invention will be better understood on reading the following description and on examining the accompanying figures. The latter are only given by way of indication and are in no way limiting to the invention. The figures show:

FIG. 1 is a diagrammatic, front view of a first embodiment of a system for determining emotional or psychological states.

FIG. 2 is a diagrammatic, cross section view taken along line A-A′ of the first embodiment of the human-multimedia interaction system of FIG. 1.

FIG. 3 is a diagrammatic, schematic view of the first embodiment of the system of FIG. 1.

FIG. 4 is a diagrammatic, schematic view of a second embodiment of a system for determining emotional or psychological states.

FIG. 5 is a diagrammatic, schematic view of a third embodiment of a system for determining emotional or psychological states.

FIG. 6 is a diagrammatic, schematic view of a fourth embodiment of a system for determining emotional or psychological states.

FIG. 7 is a flowchart of the fourth embodiment of the system.

FIG. 8 is a diagrammatic, schematic view of a fifth embodiment of a system for determining emotional or psychological states.

FIG. 9 is a diagrammatic, front view of a sixth embodiment of a system for determining emotional or psychological states.

FIG. 10 is a diagrammatic, schematic view of the sixth embodiment of the system.

FIG. 11 is a diagrammatic, schematic view of a seventh embodiment of a system for determining emotional or psychological states.

FIG. 12 is a diagrammatic, schematic view of an eighth embodiment of a system for determining emotional or psychological states.

FIG. 13 is a block diagrammatic, schematic view of a ninth embodiment of a system for determining emotional or psychological states.

FIG. 14AB are three example tables, schematic view of the ninth embodiment of a cluster engine unit of a processing device of the system.

### DETAILED DESCRIPTION

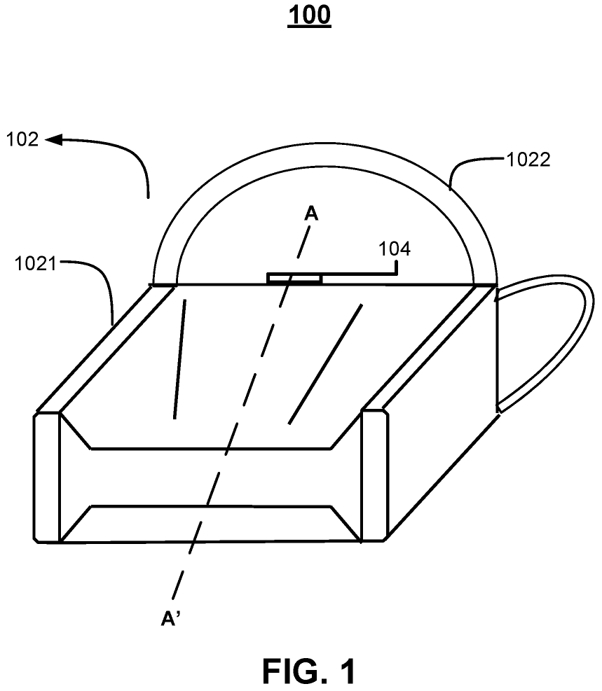

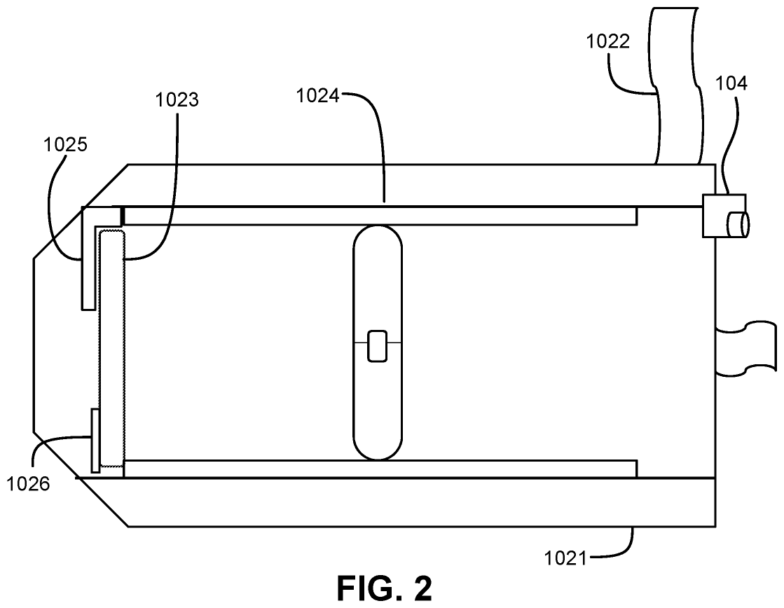

FIG. 1 illustrates a front view of the system 100 of a first embodiment. The system 100 includes a human-multimedia interaction system 102, and a detector 104. The human-multimedia interaction system 102 includes a head mounted apparatus 1021, a processing device 1025, and a storage device 1026. The head mounted apparatus 1021 includes a head holding device 1022, and a display device 1023. The head holding device 1022 is coupled to the head mounted apparatus 1021 and mounts the head mounted apparatus 1021 on a wearer's head. In the first embodiment, the processing device 1025 and the storage device 1026 are positioned in the head mounted apparatus 1021, as showing in FIG. 2.

The display device 1023 is used to receive and display video content signals. The display device 1023 may be a display panel, a display panel having an audio unit or an electrical device having a display panel and an audio unit like a mobile device or a smart phone. In the first embodiment, the display device 1023 is a display panel connected electrically to the processing device 1025. In other embodiment, the display device 1023 has a wireless communication unit and the video content signals are transmitted to the display device 1023 by wireless communication from a source. In another embodiment, the display device 1023 is connected electrically to a source, the display device 1023 receives the video content signals by a wire from the source. The source is but not limited a camera, a server, a computer, or a stored system with wire or wireless transmission. Each one of the video content signals includes at least one of the following: a video signal, an audio signal, a personal setup signal, a 3D graphics model or image (like unity3d, res S, split N, or any 3D graphics models or image file), and an image interface for interacting with the wearer.

In the first embodiment, the head mounted apparatus 1021 also includes an optic system 1024 corresponded to the display device 1023 and eyes of the wearer, as the FIG. 2. The optic system 1024 is used to adjust focuses or optic powers of the optic system 1024 for eyesight degrees of left eye and right eye of the wearer. In at least one embodiment, the display device 1023 is positioned on a surface of the optic system 1024, the optic system 1024 is configured to the wearer see the video content signals displayed by the display device 1023 and a real image at the same time. In another embodiment, the head mounted apparatus 1021 doesn't include the optic system 1024, the wearer can see clearly views displayed by the display device 1023 without the optic system 1024 or the wearer wears a glass or a contact lens for watching clearly views displayed by the display device 1023.

The processing device 1025 is coupled to the display device 1023 and the storage device 1026 by wires or wireless communication. The processing device 1025 is configured to compare at least one characteristic of the wearer detected by the detector 104 with existing data stored in the storage device 1026 or from cloud, and search at least one video content signal according to the characteristic of the wearer. The processing device 1025 is but not limited a server, a computer, or a processing chip set. In at least one embodiment, the processing device 1025 includes a wireless communication unit configured to receive the video content signals, or the characteristic of the wearer from an external computer device. The external computer device is but not limited a server, a computer, or a stored system with wire or wireless transmission function.

The storage device 1026 is coupled to the processing device 1025 and is used to receive and store the characteristic of the wearer or characteristic data of the wearer translated by the detector 104 or the processing device 1025, a plurality of the video content signals, and the existing data those identity or emotional or psychological states are known. The storage device may contain cloud-updated or locally collected data. The existing data includes at least one of the following: a cardiac parameter, a posture/activity parameter, a temperature parameter, an electroencephalography (EEG) parameter, an electro-oculography (EOG) parameter, an electromyography (EMG) parameter, an electrocardiography (ECG) parameter, a photoplethysmogram (PPG) parameter, a vocal parameter, a gait parameter, a fingerprint parameter, an iris parameter, a retina parameter, a blood pressure parameter, a blood oxygen saturation parameter, an odor parameter, and a face parameter.

The detector 104 is used to detect the characteristic of the wearer. In the first embodiment, the detector 104 is positioned on, attached to, affixed to, carried by, or incorporated in or as part of the head mounted apparatus 1021 for detecting the characteristic of the wearer, and is coupled electrically to the processing device 1025. The detector 104 is but not limits a micro-needle, a light sensor module, a part of electrodes, a pressure transducer, a biometric recognition device, microphone, a camera, a handheld device, or a wearable device. The characteristic includes at least one of the following: a cardiac parameter, a posture/activity parameter, a temperature parameter, an electroencephalography (EEG) parameter, an electro-oculography (EOG) parameter, an electromyography (EMG) parameter, an electrocardiography (ECG) parameter, a photoplethysmogram (PPG) parameter, a vocal parameter, a gait parameter, a finger print parameter, an iris parameter, a retina parameter, a blood pressure parameter, a blood oxygen saturation parameter, an odor parameter, and a face parameter.

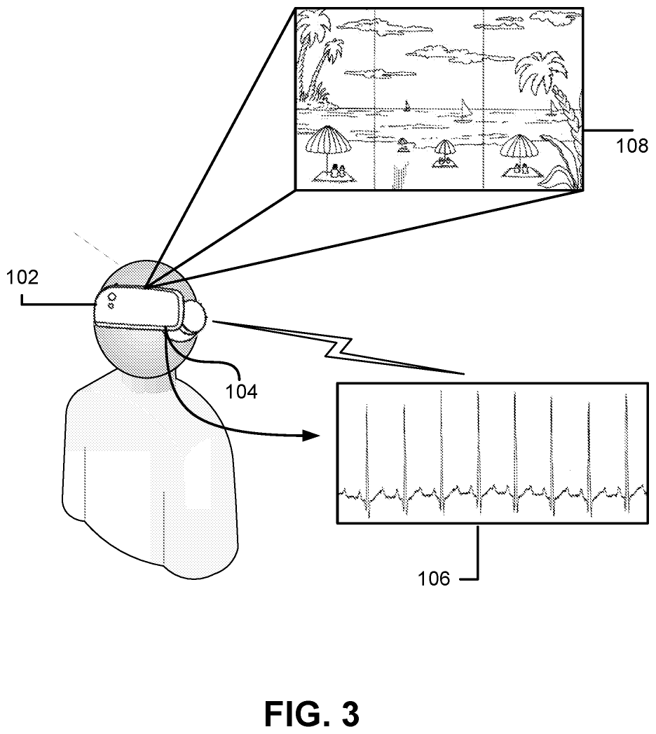

FIG. 3 shows a diagrammatic, schematic view of the first embodiment of the system 100. In the first embodiment, the head mounted apparatus 1021 includes the processing device 1025 and the storage device 1026, the detector 104 is positioned in the head mounted apparatus 1021, when the wearer wears the head mounted apparatus 1021 on the wearer's head, the detector 104 detects at least one the characteristic 106 of the wearer. The processing device 1025 receives the characteristic 106 of the wearer from the detector 104 and compares the characteristic 106 of the wearer detected by the detector 104 with the existing data those identity are known. In at least one embodiment, the processing device 1025 can analyze the characteristic 106 of the wearer, may be used Fourier Transform, and extracts one or more features of the characteristic 106 of the wearer for comparing with the existing data.

If the characteristic 106 of the wearer is different with the existing data stored in the storage device 1026 or from the cloud, that means the wearer's identity is unknown, the processing device 1025 may choose a number of the video content signals 108 stored in the storage device 1026 according to the existing data that are similar with the characteristic 106 of the wearer and transmits the video content signals 108 to the display device 1023, the wearer can choose one of the video content signals 108 displayed by the display device 1023 for setting a personalization of the wearer, the processing device 1025 sets a relation between the characteristic 106 of the wearer and the video content signal 108 chose by the wearer and the storage device 1026 received and stored the characteristic 106 of the wearer and the video content signal 108 chose by the wearer according to the characteristic 106 of the wearer.

If the characteristic 106 of the wearer is same or similar with at least one the existing data, the processing device 1025 determines that the wearer's emotional or psychological state or identity is determined or verified, the processing device 1025 transmits at least one of the video content signals 108, which may have set by the wearer, according to the characteristic 106 of the wearer to the display device 1023, and the display device 1023 displays the video content signal 108.

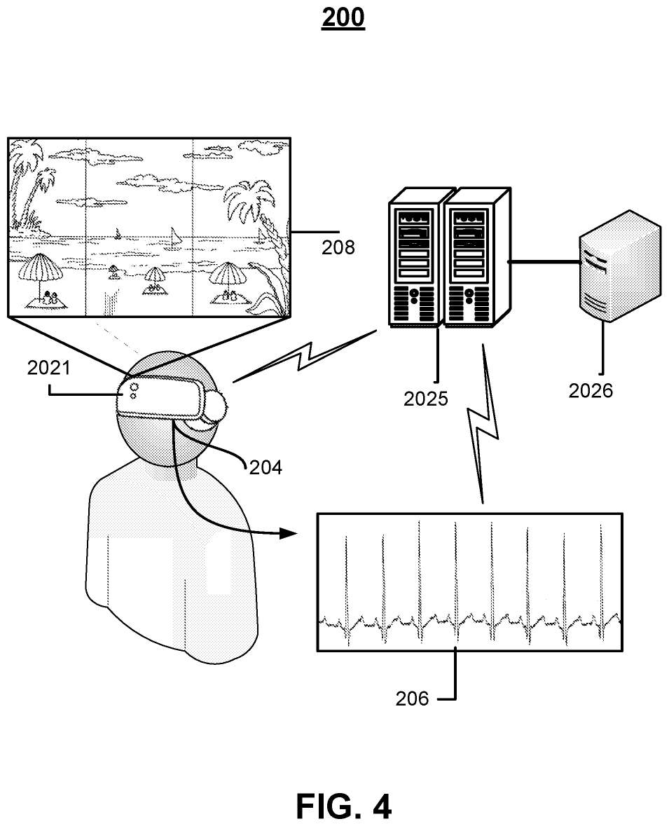

FIG. 4 is a diagrammatic, schematic view of a second embodiment of a system 200. The system 200 of the second embodiment is similar to the first embodiment except that the processing device 2025 and the storage device 2026 isn't positioned in the head mounted apparatus 2021, each one of the processing device 2025 and the head mounted apparatus 2021 includes a wireless communication unit, the processing device 2025 is coupled to the head mounted apparatus 2021 by wireless communication. The detector 204 detects at least one the characteristic 206 of the wearer, the head mounted apparatus 2021 transmits the characteristic 206 of the wearer to the processing device 2025 by wireless communication, the processing device 2025 compares the characteristic 206 of the wearer with the existing data stored in the storage device 2026 or from the cloud and transmits at least one of the video content signals 208 according to the characteristic 206 of the wearer to the head mounted apparatus 2021 by wireless communication. In at least one embodiment, the processing device 2025 is coupled electrically to the head mounted apparatus 2021 by a wire, the head mounted apparatus 2021 and the processing device 2025 transmit the characteristic 206 of the wearer and the video content signals 208 to each other by a wire.

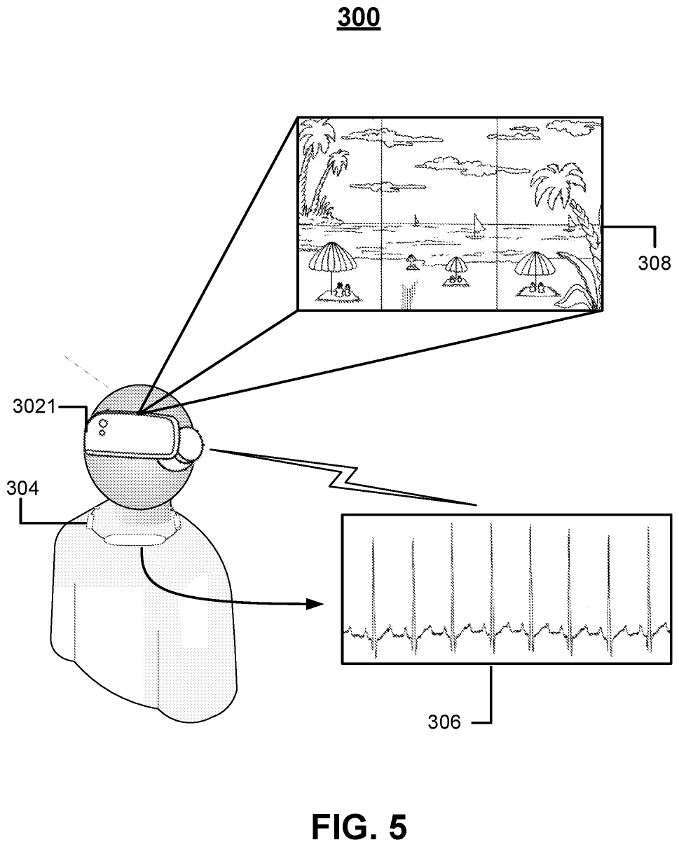

FIG. 5 is a diagrammatic, schematic view of a third embodiment of a system. The system 300 of the second embodiment is similar to the first embodiment except that the detector 304 is worn on, attached to, or mounted on the wearer, each one of the detector 304 and the head mounted apparatus 3021 includes a wireless communication unit, the detector 304 detects at least one the characteristic 306 of the wearer and transmits the characteristic 306 of the wearer to the head mounted apparatus 3021 by wireless communication. In at least one embodiment, the detector 304 is coupled electrically to the head mounted apparatus 3021 by a wire, the detector 304 transmits the characteristic 306 of the wearer to the head mounted apparatus 3021 or the process device by a wire.

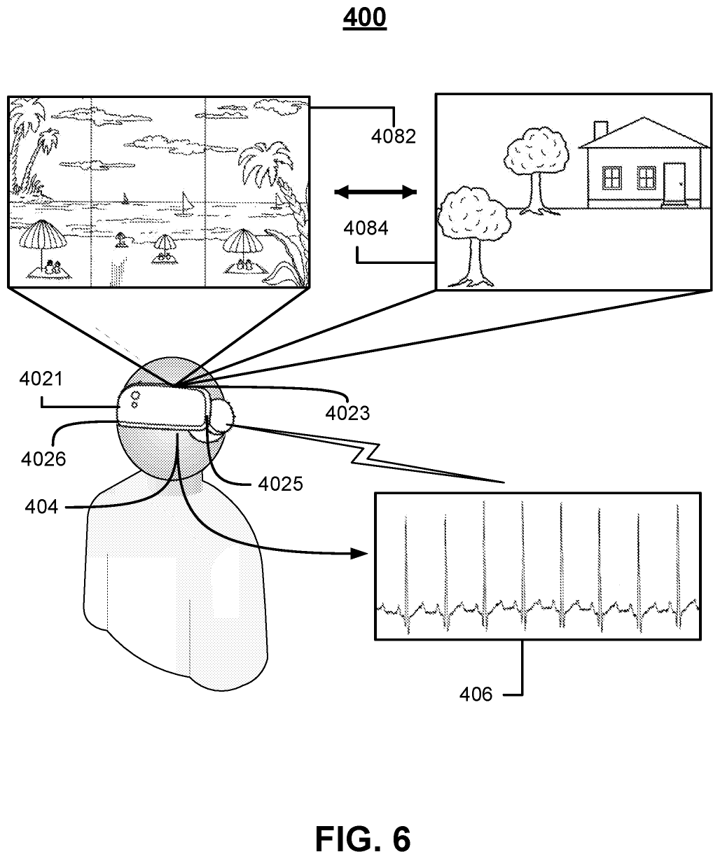

FIG. 6 is a diagrammatic, schematic view of a fourth embodiment of a system. The system 400 of the fourth embodiment is similar to the first embodiment except that the detector 404 detects at least one the characteristic 406 of the wearer, the processing device 4025 of the head mounted apparatus 4021 compares the characteristic 406 of the wearer with the existing data stored in the storage device 4026 or from the cloud and determines at least one emotional or psychological state of the wearer corresponding to the characteristic 406 of the wearer. The processing device 4025 transmits at least one the video content signal 4082 according to the emotional or psychological state of the wearer to the display device 4023. In at least one embodiment, the detector 404 can detect the characteristic 406 of the wearer at predefined intervals to observe change of the emotional or psychological state of the wearer, the processing device 4025 transmits new video content signal 4084 according to the change of the emotional or psychological state of the wearer, and the display device 4023 replaces the video content signal 4082 with new video content signal 4084.

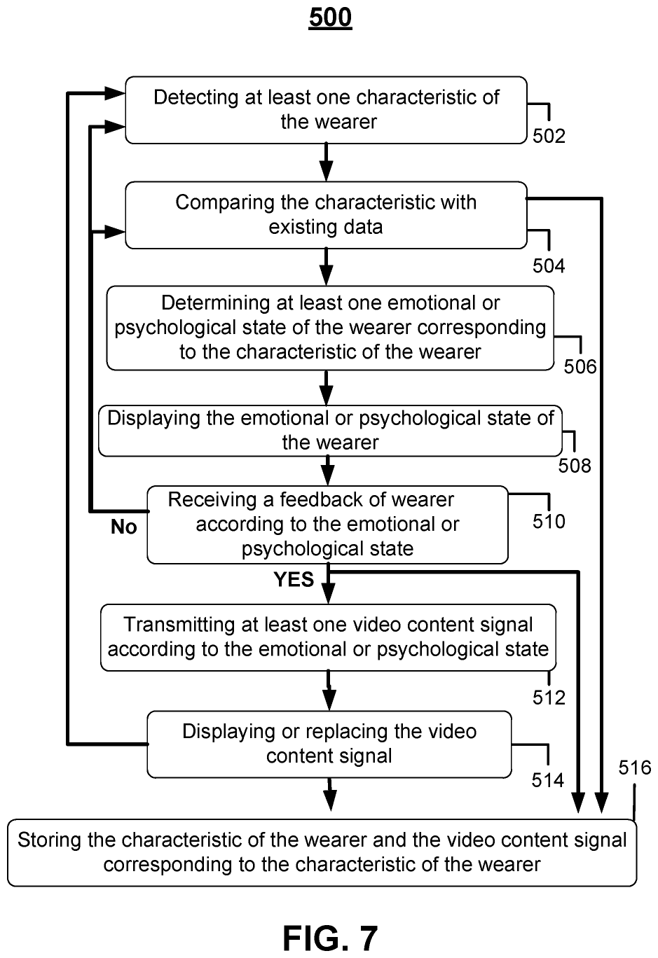

Referring to FIG. 7, a flowchart is presented in accordance with the fourth embodiment which is being thus illustrated. The example method 500 is provided by way of example, as there are a variety of ways to carry out the method. The method 500 described below can be carried out using the configurations illustrated in FIG. 6, for example, and various elements of the figure is referenced in explaining example method 500. Each block shown in FIG. 7 represents one or more processes, methods or subroutines, carried out in the example method 500. Additionally, the illustrated order of blocks is by example only and the order of the blocks can change according to the present disclosure. The example method 500 can begin at block 502.

At block 502, the detector 404 detects at least one the characteristic 406 of the wearer, the processing device 4025 receives the characteristic 406 of the wearer from the detector 404. In the fourth embodiment, the detector 404 is position on, attached to, affixed to, carried by, or incorporated in or as part of the head mounted apparatus 4021 and is coupled electrically to the processing device 4025. In at least one embodiment, the detector 404 is worn on, attached to, or mounted on the wearer, the detector 404 transmits the characteristic 406 of the wearer to the processing device 4025 of the head mounted apparatus 4021 by a wire or wireless communication.

At block 504, the processing device 4025 compares the characteristic 406 of the wearer with the existing data. In the fourth embodiment, each one of the existing data also includes an existing emotional or psychological state that emotional or psychological state is known. The storage device receives the characteristic 406 of the wearer and stores the characteristic 406 of the wearer at block 516.

At block 506, If the characteristic 406 of the wearer is determined an emotional or psychological state of the wearer and transmits the emotional or psychological state of the wearer to the display device 4023. If the characteristic 406 of the wearer is different with the existing data, the processing device 4025 may choose a number of the existing data that are similar with the characteristic 406 of the wearer and transmits the existing data included the existing emotional or psychological state to the display device 4023.

At block 508, the display device displays the emotional or psychological state of the wearer with the characteristic 406 of the wearer for the wearer or the existing data with the characteristic 406 of the wearer that the wearer can choose one of the existing data to set a personalization of the wearer.

At block 510, the processing device 4025 receives a feedback of wearer or the personalization of the wearer. If the feedback of wearer is positive, the processing device 4025 may search at least one video content signal at block 512 or the storage device receives the emotional or psychological state of the wearer with the characteristic 406 of the wearer or the personalization of the wearer and stores the emotional or psychological state of the wearer with the characteristic 406 of the wearer or the personalization of the wearer at block 516. If the feedback of wearer is negative, the detector 404 detects the characteristic 406 of the wearer one more time at the block 502 or the processing device 4025 compares the characteristic 406 of the wearer with the existing data again at the block 502.

At block 512, the processing device 4025 searches at least one video content signal 4082 according to the emotional or psychological state of the wearer and transmits the video content signal to the display device 4023.

At block 514, the display device 4023 displays the video content signal 4082 corresponding to the emotional or psychological state of the wearer. In at least one embodiment, when the display device 4023 displays the video content signal 4082, the detector 404 detects the characteristic 406 of the wearer to observe change of the emotional or psychological state of the wearer at the block 502, the processing device 4025 transmits a new video content signal 4084 according to the change of the emotional or psychological state of the wearer, and the display device 4023 replaces the video content signal 4082 with the new video content signal 4084 at block 514.

At block 516, the storage device receives the characteristic of the wearer, the video content signal corresponding to the characteristic of the wearer, and the personalization of the wearer.

In at least one embodiment, the processing device 4025 can compare the characteristic 406 of the wearer with existing data and output one of determined signals stored in the storage device 4026 at block 504. The determined signals are generated at offline trainers based on the existing data those emotional or psychological states are known and one or more data rules, each one the determined signals includes an arousal data and a valence data, the arousal data and the valence data maybe have one of arousal levels of the wearer and one of valence levels of the wearer and correspond to one or more emotional or psychological states like fear, happy, sad, content, neutral or any other emotional or psychological states of people. For example, the determined signal includes the arousal data and the valence data which have a high arousal level and a high valence level, so the arousal data and the valence data can correspond to the emotional or psychological state meant that the wearer is happy. In others embodiment, the determined signals correspond to two or more emotional or psychological states like fear, happy, sad, content, neutral or any other emotional or psychological states of people. The processing device 4025 determines an emotional or psychological state of the wearer at block 506 and searches at least one video content signal 4082 at block 512 according to the determined signals. The data rules includes, but not limit, Decision trees, Ensembles (Bagging, Boosting, Random forest), k-Nearest Neighbors algorithm (k-NN), Linear regression, Naive Bayes, Neural networks, Logistic regression, Perceptron Relevance vector machine (RVM), or Support vector machine (SVM), or any machine learning data rule.

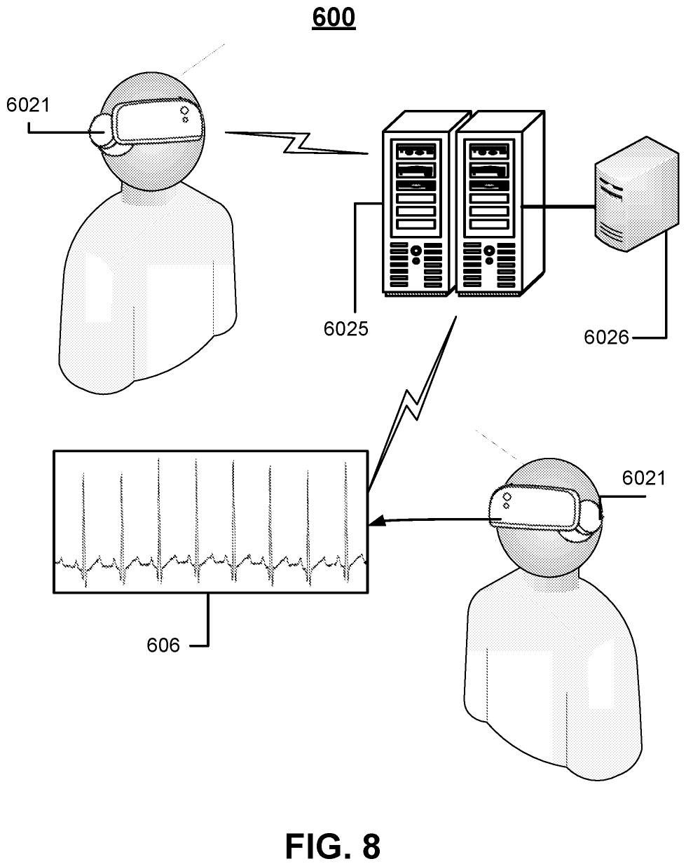

FIG. 8 is a diagrammatic, schematic view of a fifth embodiment of a system 600. The system 600 of the fifth embodiment is similar to the second embodiment except that the system 600 is used to communicate with at least one user who wears the head mounted apparatus 6021 in an augmented, virtual environment or an internet environment. The processing device 6025 compares the characteristic 606 of the wearer with the existing data stored in the storage device 6026 or from the cloud and determines the wearer's identity is verified or the wearer's emotional or psychological state, the processing device 6025 searches at least one video content signal according to the characteristic 606 of the wearer. In the fifth embodiment, the video content signal includes a personal setup signal set by the wearer according to a face parameter and a body parameter of the wearer, the processing device 6025 can sets a virtual body video signal according to the personal setup signal of the video content signal and transmits the virtual body video signal of the wearer to the head mounted apparatus 6021 for communicating with each other in the virtual environment or the internet environment. In at least one embodiment, the detector of the head mounted apparatus 6021 detects a change of the face parameter of the wearer like facial expressions, and the processing device 6025 receives the change of the face parameter of the wearer for changing a facial expression of the virtual body video signal of the wearer for instance.

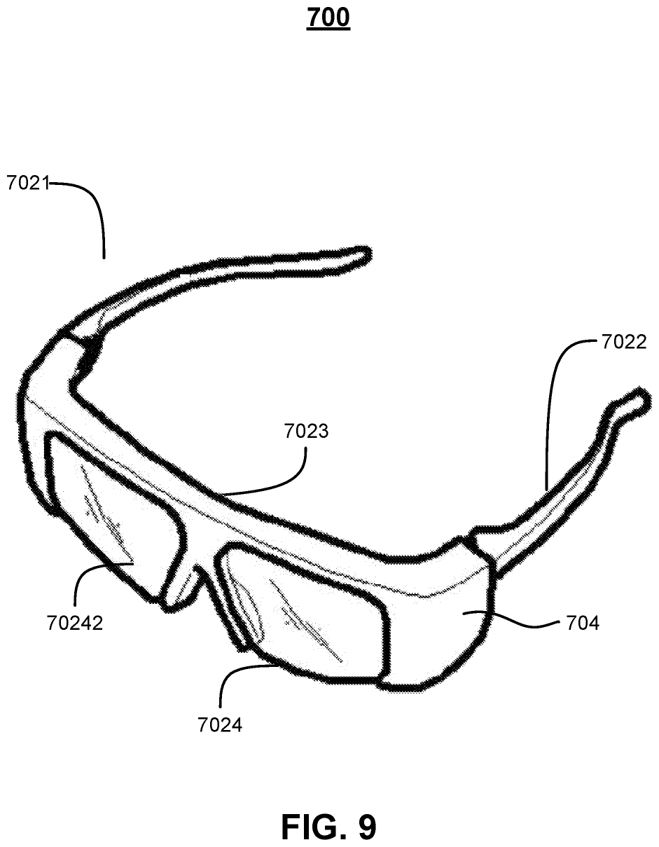

FIG. 9 is a diagrammatic, front view of a sixth embodiment of a system 700. The system 700 of the sixth embodiment is similar to the second embodiment except that the head mounted apparatus 7021 is a glass including the head holding device 7022, the display device 7023, the optic system 7024, and the detector 704. The optic system 7024 includes two lenses 70242 corresponding to a right eye and a left eye of the wearer. Each one of lenses 70242 is transparent, the display device 7023 is positioned on a pair or all of an optic face of one of the lenses 70242, the optic face is a front surface of the lens 70242 or a back surface of the lens 70242.

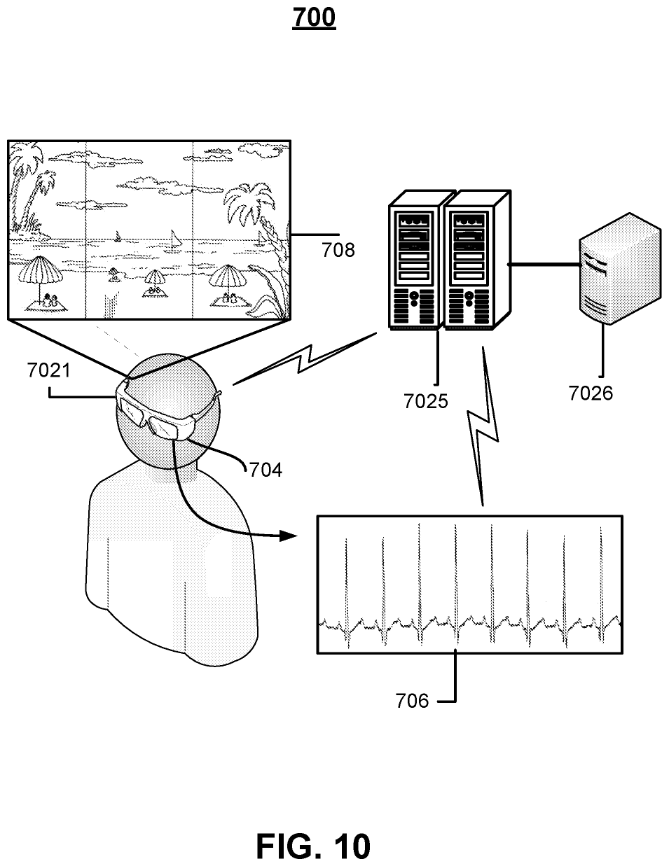

FIG. 10 is a diagrammatic, schematic view of a sixth embodiment of a system 700. Each one of the processing device 7025 and the head mounted apparatus 7021 includes a wireless communication unit, the processing device 7025 is coupled to the head mounted apparatus 7021 by wireless communication. The detector 704 detects at least one the characteristic 706 of the wearer, the head mounted apparatus 7021 transmits the characteristic 706 of the wearer to the processing device 7025 by wireless communication, the processing device 7025 compares the characteristic 706 of the wearer with the existing data stored in the storage device 7026 or from the cloud and transmits at least one of the video content signals 708 according to the characteristic 706 of the wearer to the head mounted apparatus 7021 by wireless communication, the wearer may see the video content signals 708 displayed by the display device 7023 and a real image at the same time.

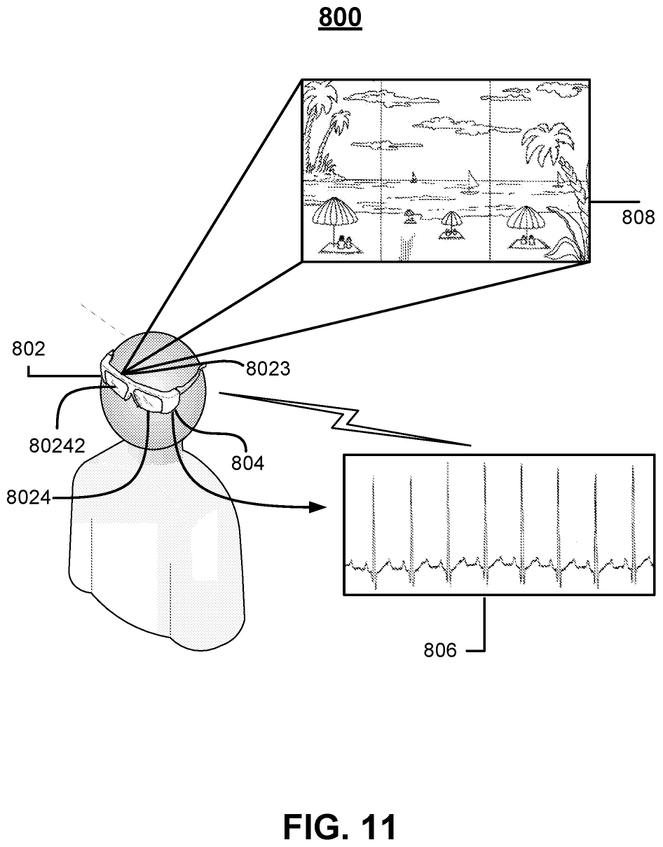

FIG. 11 is a diagrammatic, schematic view of a seventh embodiment of a system 800. The system 800 of the seventh embodiment is similar to the first embodiment except that the head mounted apparatus 8021 is a glass including the head holding device 8022, the display device 8023, the optic system 8024, and the detector 804. The optic system 8024 includes two lenses 80242 corresponding to a right eye and a left eye of the wearer. Each one of lenses 80242 is transparent, the display device 8023 is positioned on a pair or all of an optic face of one of the lenses 80242, the optic face is a front surface of the lens 80242 or a back surface of the lens 80242. The wearer may see the video content signals 808 displayed by the display device 8023 and a real image at the same time.

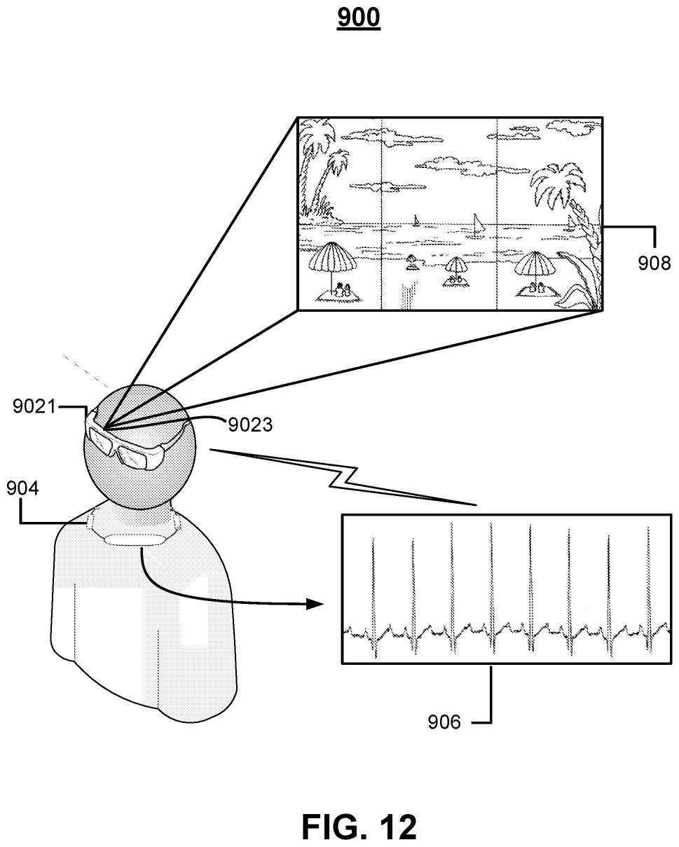

FIG. 12 is a diagrammatic, schematic view of an eighth embodiment of a system 900. The system 900 of the eighth embodiment is similar to the seventh embodiment except that the detector 904 is worn on, attached to, or mounted on the wearer, each one of the detector 904 and the head mounted apparatus 9021 includes a wireless communication unit, the detector 904 detects at least one the characteristic 906 of the wearer and transmits the characteristic 906 of the wearer to the head mounted apparatus 9021 by wireless communication. The processing device transmits at least one of the video content signals 908 according to the characteristic 906 of the wearer to the head mounted apparatus 9021 by wireless communication, the wearer may see the video content signals 908 displayed by the display device 9023 and a real image at the same time.

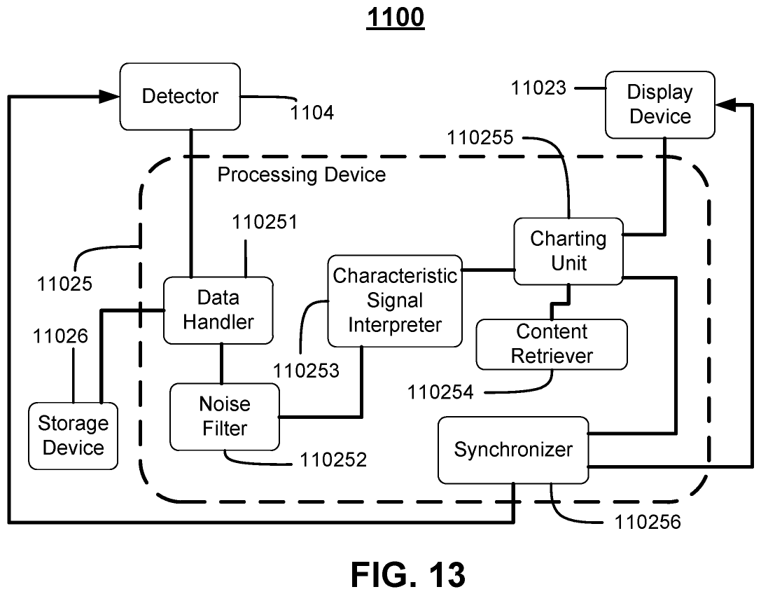

FIG. 13 is a schematic block diagram view of a ninth embodiment of a system 1100. The system 1100 of the ninth embodiment is similar to the one embodiment except that the processing device 11025 includes a data handler 110251, a noise filter 110252, a characteristic signal interpreter 110253, a content retriever 110254, a cluster engine unit 110255, and a synchronizer 110256. In the ninth embodiment, the detector 1104 detects at least one characteristic of the wearer, the data handler 110251 receives the characteristic of the wearer from the detector 1104 for outputting a signal of the characteristic of the wearer and stores the characteristic of the wearer in the storage device 11026, the noise filter 110252 can perform the signal of the characteristic of the wearer to remove unwanted frequency components. The characteristic signal interpreter 110253 receives the characteristic of the wearer via the noise filter 110252 from the data handler 110251, catches at least one feature of the characteristic of the wearer for comparing with the existing data those emotional or psychological states are known, and determines an emotional or psychological state of the wearer. The characteristic signal interpreter 110253 transmits the emotional or psychological state of the wearer to the cluster engine unit 110255. In the ninth embodiment, the cluster engine unit 110255 may be a graphical user interface (GUI) parser and is configured to provide a user interface displayed on the display device 11023. In others embodiment, the cluster engine unit 110255 is used to provide suitable views of the characteristic of the wearer.

The content retriever 110254 is configured to record one or more segments of the video content signal with timestamp of the video content signal as timestamps indicating when images of the segment were captured. In the ninth embodiment, the video content signal includes one or more segments, each one of the segments maybe affect the emotional or psychological state of the wearer or the characteristic of the wearer, the content retriever 110254 can record all of the segments of the video content signal with timestamp of the video content signal and transmits to the cluster engine unit 110255.

In the ninth embodiment, the data handler 110251 also records timestamp of the characteristic and the characteristic signal interpreter 110253 transmits the emotion states of the wearer with the timestamp of the characteristic to the cluster engine unit 110255, the cluster engine unit 110255 receives the emotion states of the wearer with the timestamp of the characteristic and the segments of the video content signal with the timestamp of the video content signal, the cluster engine unit 110255 can list, arrange, merge, or combine the emotions state of the wearer and the segments of the video content signal according to the timestamp of the video content signal and the timestamp of the physiological characteristic.

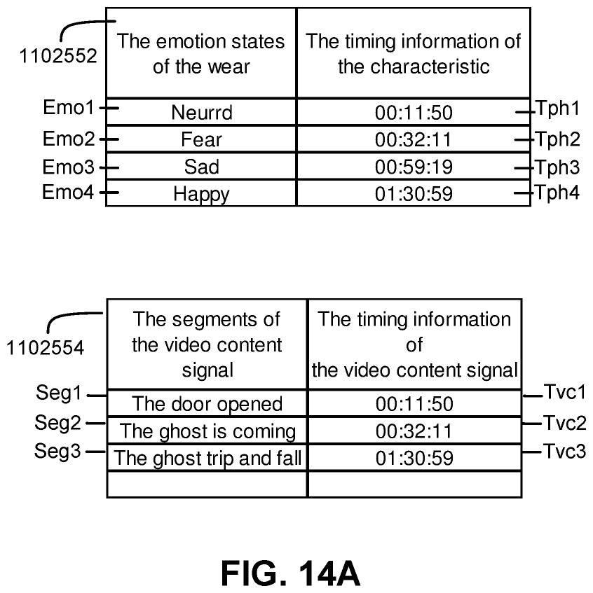

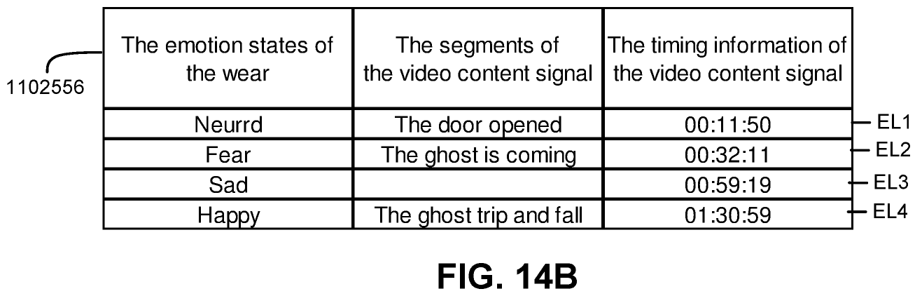

For example, FIG. 14A shows an emotion table 1102552 for the emotion states of the wearer with the timestamp of the characteristic and a content table 1102554 for the segments of the video content signal with the timestamp of the video content signal, the cluster engine unit 110255 receives the emotion states of the wearer with the timestamp of the characteristic and the segments of the video content signal with the timestamp of the video content signal, and outputs the emotion table 1102552 and the content table 1102554, the emotion table 1102552 includes four emotion state Emo1, Emo2, Emo3, and Emo4 and timestamp Tph1, Tph2, Tph3, and Tph4. Each one the emotion state is corresponded to each one the timestamp. For example, the emotion state Emo1 is determined according to the characteristic of the wearer detected at a time that is recorded to the timestamp Tph1, so the emotion state Emo1 is corresponded to the timestamp Tph1. the content table 1102554 is similar to the emotion table 1102552 and includes three segments Seg1, Seg2, and Seg3 and timestamp Tvc1, Tvc2, and Tvc3. The segments Seg1 is corresponded to the timestamp Tvc1 and so on.

When the cluster engine unit 110255 lists, arranges, merges, or combines the emotions state of the wearer and the segments of the video content signal, the cluster engine unit 110255 compares the timestamp Tph1, Tph2, Tph3, and Tph4 with the timestamp Tvc1, Tvc2, and Tvc3. If each two the timestamp is the same, for example, the timestamp Tph1 is same with the timestamp Tvc1, the cluster engine unit 110255 can determine the emotion state Emo1 is corresponded to the segment Seg1 and outputs an emotion item EL1 in an emotion retriever list 1102556 as the FIG. 14B. If at least one the timestamp Tph1, Tph2, Tph3, and Tph4 is different to all the timestamp Tvc1, Tvc2, and Tvc3, like the emotion state Emo3, that means the emotion state Emo3 doesn't corresponded to any the segments of the video content signal, the cluster engine unit 110255 outputs the emotion item EL3 in the emotion retriever list 1102556 and transmits the emotion retriever list 1102556 to the synchronizer 110256, and vice versa.

In at least one embodiment, the cluster engine unit 110255 outputs the emotion item at every moment of the segments of the video content signal and transmits the emotion item to the synchronizer 110256, the synchronizer 110256 can control quality that comparing the characteristic of the wearer with the existing data those emotional or psychological states are known or relativity between the emotion state of wearer and the segments of the video content signal, and feedbacks to the detector 1104 and the display device 11023 for determining the emotion state of the wearer one more time or not. In others embodiment, the cluster engine unit 110255 catches at least one the emotions state of the wearer to show on the user interface displayed by the display device 11023 for the wearer.

The synchronizer 110256 is configured to control quality that comparing the characteristic of the wearer with the existing data those emotional or psychological states are known or relativity between the emotion state of wearer and the segments of the video content signal. In the ninth embodiment, the cluster engine unit 110255 lists, arranges, merges, or combines the emotions state of the wearer and the segments of the video content signal and transmits to the synchronizer 110256, the synchronizer 110256 determines quality of relativity between the emotion state of wearer and the segments of the video content signal. If the quality is low, the synchronizer 110256 feedbacks to the detector 1104 and the display device 11023 for determining the emotion state of the wearer one more time. On the other hand, if the quality is good, the synchronizer 110256 feedbacks to the display device 11023 for changing the video content signal and the detector 1104 for detecting whether the emotional or psychological state of the wearer make a change according to changing the video content signal.

In at least one embodiment, the synchronizer 110256 is coupled to the noise filter 110252 or the characteristic signal interpreter 110253 for controlling quality of the characteristic of the wearer or similarity between the characteristic of the wearer and the existing data.

In at least one embodiment, the detector is positioned on the head mounted apparatus and is configured to detect at least one feature of eyes of the wearer, for example, detecting eyesight degrees of eyes of the wearer. The feature of eyes of the user includes, but not limit, eyesight degrees of eyes of a user, eye movement, blinking frequency or the like.

Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

The invention claimed is:

1. A system, comprising:
a head mounted apparatus;
a detector detecting at least one characteristic of a user;
a storage device storing characteristic date of the user;
a processing device coupled to the storage device, the detector, and the head mounted apparatus.

2. The system as claimed in claim 1, wherein system includes a display device coupled to the processing device.

3. The system as claimed in claim 1, wherein the processing device compares the characteristic of a user with existing data stored in the storage device or from the cloud.

4. The system as claimed in claim 1, wherein the processing device compares the characteristic of a user with the existing data to determine emotional or psychological state of the user.

5. The system as claimed in claim 1, wherein the detector is coupled to the head mounted apparatus.

6. The system as claimed in claim 1, wherein the detector is configured to detect at least one feature of eyes of a user.

7. The system as claimed in claim 1, wherein the detector is positioned on the head mounted apparatus and is configured to detect at least one feature of eyes of a user.

8. The system as claimed in claim 1, wherein the detector includes a first wireless communication unit and the processing device include a second wireless communication unit.

9. The system as claimed in claim 2, wherein the storage device stores a plurality of video content signals.

10. The system as claimed in claim 2, wherein the processing device determines emotional or psychological state of the user according to at least one character is tic of a user.

11. The system as claimed in claim 9, wherein the processing device determines emotional or psychological state of the user according to at least one characteristic of a user and transmits one of the video content signals to the display device according to the emotional or psychological state.

12. The system as claimed in claim 2, where in the processing device includes a data handler, a content retriever, and a cluster engine unit connected to the data handler and the cluster engine unit and configured to receive data produced by the data handler and the content retriever.

13. A method of distinguishing personal characteristics for a system, comprising:
detecting, by a detector, at least one characteristic of a user;
comparing, by a processing device, the characteristic of a user with the existing data;
determining, by the processing device, a personal characteristic of a user according to the characteristic of a user.

14. The method of distinguishing personal characteristics as claimed in claim 13, wherein the processing device compares the characteristic of a user with the existing data to determine emotional or psychological state of the user.

15. The method of distinguishing personal characteristics as claimed in claim 13, wherein the processing device compares the characteristic of a user with the existing data to determine the user's identity.

16. The method of distinguishing personal characteristics as claimed in claim 13, wherein the system includes a display device to displaying one of the video content signals for detecting the characteristic of a user.

17. The method of distinguishing personal characteristics as claimed in claim 13, wherein the detector is positioned on the head mounted apparatus and is configured to detect at least one feature of eyes of the user.

18. The method of distinguishing personal characteristics as claimed in claim 14, wherein the system includes a display device to display the video content signal, the detector detects the characteristic of a user for observing change of emotional or psychological state of the user, and the display device replaces the video content signal according to the change of the emotional or psychological state.

19. The method of distinguishing personal characteristics as claimed in claim 16, where in the processing device records a segment of the video content signal, and the personal characteristic of a user with timestamp of the video content signal and timestamp of the personal characteristic of a user.

20. The method of distinguishing personal characteristics as claimed in claim 18, where in the processing device records a segment of the video content signal, and the emotional or psychological state with timestamp of the video content signal and timestamp of the emotional or psychological state.
